(19) United States
(12) Patent Application Publication
(10) Pub. No.: US 2026/0324947 A1
(43) Pub. Date: Oct. 22, 2026

(54) ENDOSCOPIC SUPPORT DEVICE, ENDOSCOPIC EXAMINATION SUPPORT METHOD, AND RECORDING MEDIUM

(71) Applicant: NEC Corporation, Tokyo (JP)

(72) Inventor: Yasuo OMI, Tokyo (JP)

(73) Assignee: NEC Corporation, Tokyo (JP)

(21) Appl. No.: 19/491,106

(22) PCT Filed: Jun. 28, 2023
(86) PCT No.: PCT/JP2023/023905

§ 371 Date: Dec. 8, 2025

(51) Int. Cl. A61B 1/00

(52) U.S. Cl. CPC A61B 1/000096; A61B 1/00042; A61B 1/00045

(57) ABSTRACT

In an endoscopic examination support device, by using a parameter obtained based on an endoscopic image containing a subject in a body cavity, an image generation means generates a first image group containing a plurality of arbitrary-viewpoint endoscopic images corresponding to respective viewpoint positions different from each other, or generates a second image group containing at least one arbitrary-viewpoint endoscopic image corresponding to a viewpoint position different from a viewpoint position of the endoscopic image. An inference means performs inference processing related to the subject based on the first image group or based on the endoscopic image and the second image group. The endoscopic examination support device is designed to support the decision making of medical doctors in diagnosing lesions.

### TECHNICAL FIELD

The present disclosure relates to a technique that is utilizable to present information for supporting endoscopic examinations.

### BACKGROUND ART

Techniques for supporting endoscopic examinations have been proposed.

Specifically, for instance, Patent Literature 1 discloses a technique in which, in an endoscope device including a parent endoscope and a child endoscope, an endoscopic image obtained from the child endoscope is corrected, and diagnostic support information including the presence or absence of a lesion or the like contained in a corrected endoscopic image is output by inputting the corrected endoscopic image to a diagnostic support learning model.

### PRECEDING TECHNICAL REFERENCES

### Patent Document

Patent Document 1: Japanese Laid-open Patent Publication No. 2022-175037

### SUMMARY

### Problem to be Solved by the Invention

However, Patent Literature 1 does not particularly disclose an approach for obtaining information related to a lesion from a plurality of endoscopic images corresponding to a plurality of respective viewpoints different from each other. Hence, according to the technique disclosed in Patent Literature 1, a problem arises that accuracy of the information that is related to the lesion and is obtained from the endoscopic image deteriorates, for instance.

One object of the present disclosure is to provide an endoscopic examination support device capable of improving accuracy of information that is related to a lesion and is obtained from an endoscopic image.

### Means for Solving the Problem

According to an example aspect of the present disclosure, there is provided an endoscopic examination support device including:
image generation means configured to, by using a parameter obtained based on an endoscopic image containing a subject in a body cavity, generate a first image group containing a plurality of arbitrary-viewpoint endoscopic images corresponding to respective viewpoint positions different from each other, or generate a second image group containing at least one arbitrary-viewpoint endoscopic image corresponding to a viewpoint position different from a viewpoint position of the endoscopic image; and
inference means configured to perform inference processing related to the subject based on the first image group or based on the endoscopic image and the second image group.

According to another example aspect of the present disclosure, there is provided an endoscopic examination support method including:
by using a parameter obtained based on an endoscopic image containing a subject in a body cavity, generating a first image group containing a plurality of arbitrary-viewpoint endoscopic images corresponding to respective viewpoint positions different from each other, or generating a second image group containing at least one arbitrary-viewpoint endoscopic image corresponding to a viewpoint position different from a viewpoint position of the endoscopic image; and
performing inference related to the subject based on the first image group or based on the endoscopic image and the second image group.

According to still another example aspect of the present disclosure, there is provided a recording medium storing a program, the program causing a computer to perform a process including:
processing of, by using a parameter obtained based on an endoscopic image containing a subject in a body cavity, generating a first image group containing a plurality of arbitrary-viewpoint endoscopic images corresponding to respective viewpoint positions different from each other, or generating a second image group containing at least one arbitrary-viewpoint endoscopic image corresponding to a viewpoint position different from a viewpoint position of the endoscopic image; and
processing of performing inference related to the subject based on the first image group or based on the endoscopic image and the second image group.

### Effect of the Invention

According to the present disclosure, it is possible to improve the accuracy of information that is related to the lesion and is obtained from the endoscopic image.

### BRIEF DESCRIPTION OF THE DRAWINGS

FIG. 1 is a diagram illustrating a schematic configuration example of an endoscopic examination system according to the present disclosure.

FIG. 2 is a block diagram illustrating a hardware configuration example of an endoscopic examination support device according to the present disclosure.

FIG. 3 is a block diagram illustrating a functional configuration example of the endoscopic examination support device according to the present disclosure.

FIG. 4 is a diagram for describing an example of a parameter used for processing of the endoscopic examination support device according to the present disclosure.

FIG. 5 is a diagram for describing a functional configuration example of inference processing units.

FIG. 6 is a flowchart illustrating an example of the processing performed in the endoscopic examination support device according to the present disclosure.

FIG. 7 is a block diagram illustrating another functional configuration example of the endoscopic examination support device according to the present disclosure.

FIG. 8 is a diagram for describing another functional configuration example of an inference processing unit.

FIG. 9 is a flowchart illustrating another example of the processing performed in the endoscopic examination support device according to the present disclosure.

FIG. 10 is a block diagram illustrating still another functional configuration example of the endoscopic examination support device according to the present disclosure.

FIG. 11 is a flowchart illustrating still another example of the processing performed in the endoscopic examination support device according to the present disclosure.

### EXAMPLE EMBODIMENTS

Hereinafter, preferred example embodiments of the present disclosure will be described with reference to the drawings.

### First Example Embodiment

### System Configuration

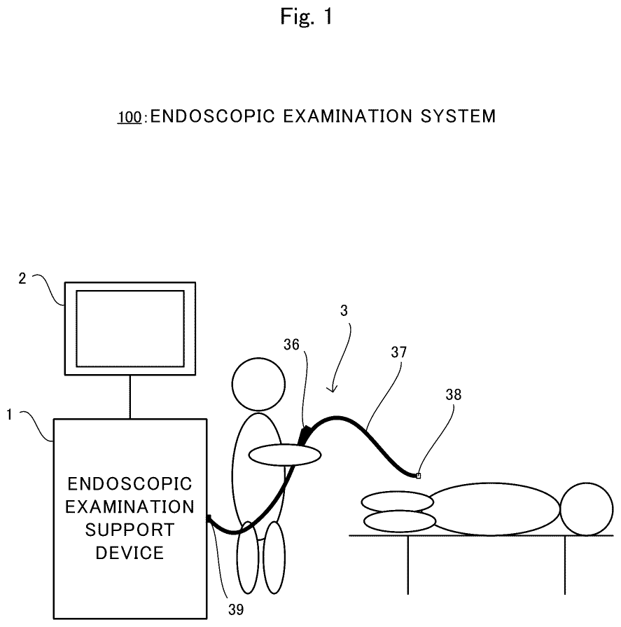

FIG. 1 is a diagram illustrating a schematic configuration example of an endoscopic examination system according to the present disclosure. As illustrated in FIG. 1, an endoscopic examination system 100 includes an endoscopic examination support device 1, a display device 2, and an endoscopic scope 3 that is connected to the endoscopic examination support device 1.

The endoscopic examination support device 1 acquires, from the endoscopic scope 3, a video (hereinafter, also referred to as an endoscopic video) containing time-series images that are obtained by imaging a subject during an endoscopic examination, and causes the display device 2 to display various display images for checking by an operator, such as a doctor, performing the endoscopic examination. Specifically, the endoscopic examination support device 1 acquires, from the endoscopic scope 3, a video of an inside of a large intestine obtained during the endoscopic examination, as the endoscopic video. The endoscopic examination support device 1 extracts one endoscopic image from the endoscopic video, and generates a plurality of arbitrary-viewpoint endoscopic images corresponding to respective viewpoint positions different from each other, by using parameters that are obtained based on the extracted one endoscopic image. The endoscopic examination support device 1 acquires, based on the plurality of arbitrary-viewpoint endoscopic images, information relating to a lesion in the one endoscopic image extracted from the endoscopic video. The endoscopic examination support device 1 is usable for supporting the doctor in decision making that relates to a diagnosis of the lesion.

The display device 2 includes, for instance, a liquid crystal monitor. The display device 2 displays the display images or the like output from the endoscopic support device 1.

The endoscopic scope 3 mainly includes an operation unit 36 for the operator to perform air supply, water supply, angle adjustment, and input of a capture instruction and the like, a shaft 37 that has flexibility and is inserted into an organ, which is an examination target, of an examination subject, a distal end portion 38 that incorporates an endoscopic camera, such as an ultra-small imaging element, and a connection unit 39 for connecting to the endoscopic examination support device 1.

### Hardware Configuration

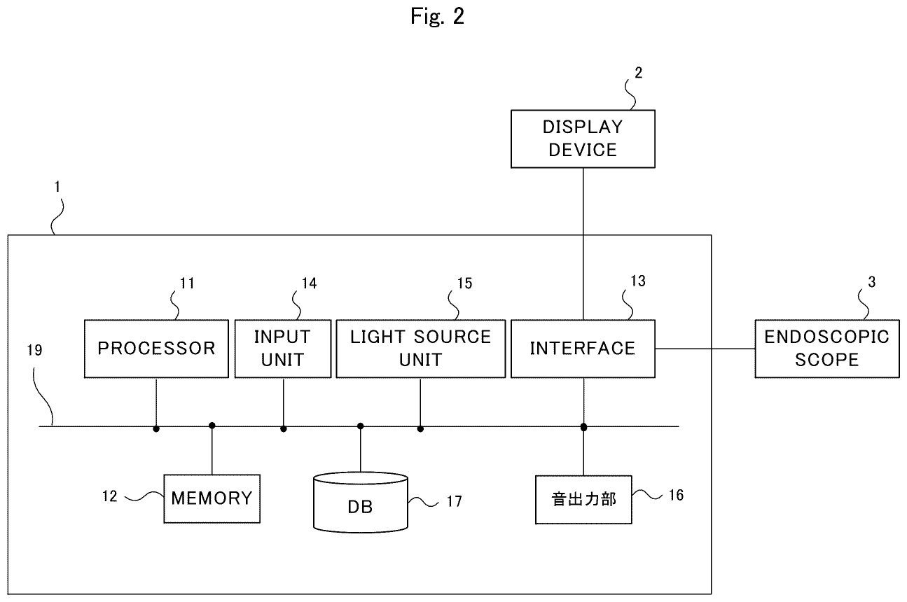

FIG. 2 is a block diagram illustrating a hardware configuration example of the endoscopic examination support device according to the present disclosure. The endoscopic examination support device 1 mainly includes a processor 11, a memory 12, an interface 13, an input unit 14, a light source unit 15, a sound output unit 16, and a database (hereinafter, referred to as “DB”) 17. These elements are connected via a data bus 19.

The processor 11 executes predetermined processing by executing a program or the like stored in the memory 12. The processor 11 is a processor, such as a central processing unit (CPU), a graphics processing unit (GPU), and a tensor processing unit (TPU). The processor 11 may be configured with a plurality of processors. The processor 11 is an example of a computer. The processor 11 performs processing of acquiring information related to the lesion from the plurality of arbitrary-viewpoint endoscopic images corresponding to the plurality of respective viewpoints different from each other, and other processing.

The memory 12 is configured with various volatile memories that are used as a working memory, such as a random-access memory (RAM) and a read-only memory (ROM), and a nonvolatile memory that stores information necessary for processing of the endoscopic examination support device 1. The memory 12 may include an external storage device, such as a hard disk, connected to or incorporated in the endoscopic examination support device 1, or may include a storage medium, such as a removable flash memory and a disk/disc medium. The memory 12 stores a program for the endoscopic examination support device 1 to execute each piece of processing in the present example embodiment.

The memory 12 temporarily stores the sequential endoscopic video that is captured by the endoscopic scope 3 in the endoscopic examination under control of the processor 11.

The interface 13 performs an interface operation between the endoscopic examination support device 1 and an external device. For instance, the interface 13 supplies display images generated by the processor 11 to the display device 2. The interface 13 supplies illumination light generated by the light source unit 15 to the endoscopic scope 3. The interface 13 supplies, to the processor 11, electric signals indicating the endoscopic video that is supplied from the endoscopic scope 3. The interface 13 supplies the endoscopic image extracted from the endoscopic video to the processor 11. The interface 13 may be a communication interface, such as a network adapter for performing wired or wireless communication with the external device, or may be a hardware interface in compliance with a universal serial bus (USB) standard, a serial AT attachment (SATA) standard, or the like.

The input unit 14 generates an input signal conforming to an operation of the operator. The input unit 14 includes, for instance, at least one device of a button, a touch panel, a remote controller, a foot switch, and a voice input device. The light source unit 15 generates the light to be supplied to the distal end portion 38 of the endoscopic scope 3. The light source unit 15 may also incorporate a pump or the like for delivering water or air to be supplied to the endoscopic scope 3. The sound output unit 16 outputs sound under the control of the processor 11.

The DB 17 stores the endoscopic video or the like acquired in a past endoscopic examination of the examination subject. The DB 17 may include an external storage device, such as a hard disk connected to or incorporated in the endoscopic examination support device 1, or may include a storage medium, such as a removable flash memory. Instead of including the DB 17 in the endoscopic examination system 100, the DB 17 may be provided on an external server or the like, and related information may be acquired from the server by communication.

The endoscopic examination support device 1 may include a sensor, such as a magnetic sensor, capable of measuring rotation and translation of the endoscopic camera.

### Functional Configuration

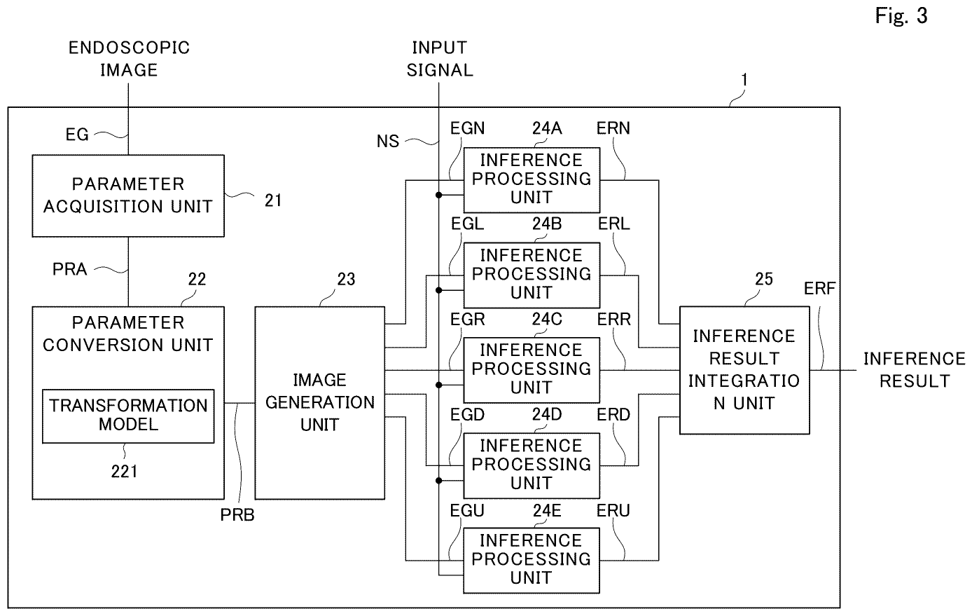

FIG. 3 is a block diagram illustrating a functional configuration example of the endoscopic examination support device according to the present disclosure. As illustrated in FIG. 3, the endoscopic examination support device 1 includes a parameter acquisition unit 21, a parameter conversion unit 22, an image generation unit 23, five inference processing units 24A to 24E, and an inference result integration unit 25.

The parameter acquisition unit 21 has a function of parameter acquisition means. The parameter acquisition unit 21 acquires a parameter PRA representing a viewpoint position and a line-of-sight direction that correspond to each pixel of an endoscopic image EG, and outputs the acquired parameter PRA to the parameter conversion unit 22.

The endoscopic image EG is desirably, for instance, an image obtained at a timing in a case where a capture button of the input unit 14 is operated. The endoscopic image EG may be, for instance, images extracted from the endoscopic video at regular intervals. The endoscopic image EG may be, for instance, an image having the best image quality among endoscopic images contained in an endoscopic video for a predetermined period from before the operation of the capture button of the input unit 14 until after the operation. The following description is given on the assumption that a tissue matching the lesion, as the subject in a body cavity, is contained in the endoscopic image EG, unless otherwise specified.

A specific example of processing performed in the parameter acquisition unit 21 will now be described.

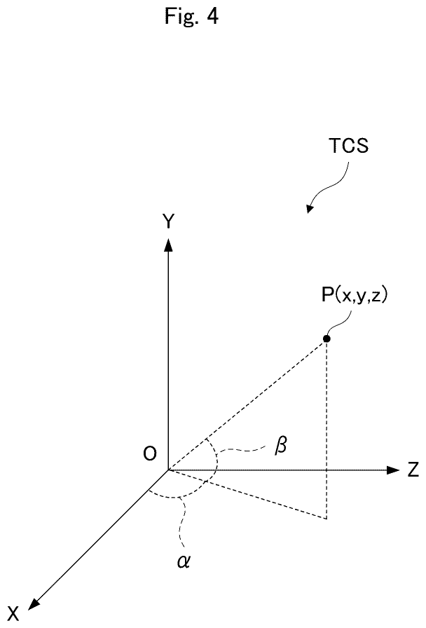

In response to input of the endoscopic image EG, the parameter acquisition unit 21 sets a three-dimensional cartesian coordinate system (hereinafter, also referred to as a camera coordinate system) TCS as illustrated in FIG. 4, for instance. FIG. 4 is a diagram for describing a parameter used for the processing of the endoscopic examination support device according to the present disclosure.

Specifically, the parameter acquisition unit 21 sets, as an origin O of the camera coordinate system TCS, a position of the endoscopic camera in a case where the endoscopic image EG is acquired. The parameter acquisition unit 21 sets a horizontal direction viewed from the origin O as an X-axis direction of the camera coordinate system TCS. The parameter acquisition unit 21 sets a vertical direction viewed from the origin O as a Y-axis direction of the camera coordinate system TCS. The parameter acquisition unit 21 sets a depth direction viewed from the origin O as a Z-axis direction of the camera coordinate system TCS.

The parameter acquisition unit 21 acquires coordinates P(x, y, z) in the camera coordinate system TCS, as a parameter representing the viewpoint position corresponding to each pixel of the endoscopic image EG (see FIG. 4). The parameter acquisition unit 21 acquires an azimuth angle α and an angle of elevation/depression β viewed from the origin O at the coordinates P, as a parameter representing the line-of-sight direction corresponding to each pixel of the endoscopic image EG (see FIG. 4). The parameter acquisition unit 21 acquires, as the parameter PRA, a five-dimensional parameter (x, y, z, α, β) corresponding to each pixel of the endoscopic image EG, and outputs the acquired parameter PRA to the parameter conversion unit 22.

The parameter acquisition unit 21 can acquire values of x and y of the parameter PRA as coordinate values corresponding to a position of each pixel contained in the endoscopic image EG. The parameter acquisition unit 21 can acquire a value of z of the parameter PRA as, for instance, a coordinate value corresponding to a processing result that is obtained by applying processing, such as depth estimation, to the endoscopic image EG. The parameter acquisition unit 21 may acquire the value of z of the parameter PRA as a coordinate value same as an average value, a representative value, a predetermined value, or the like corresponding to the position of each pixel contained in the endoscopic image EG. In a case where the value of z of the parameter PRA is calculated, the parameter acquisition unit 21 may use, for instance, a technique disclosed in Pierluigi Zama Ramirez, et. al, “Unsupervised Novel View Synthesis from a Single Image” (hereinafter, referred to as Non Patent Literature DH 1). The azimuth angle α of the parameter PRA can be rephrased as an angle in a left-right direction or an angle in the horizontal direction viewed from the origin O at the coordinates P. The angle of elevation/depression β of the parameter PRA can be rephrased as an angle in an up-down direction or an angle in the vertical direction viewed from the origin O at the coordinates P.

The parameter conversion unit 22 has a function of parameter conversion means. The parameter conversion unit 22 includes a transformation model 221 that has been learned. The transformation model 221 is configured as a machine learning model trained to output a four-dimensional parameter representing a color and volume density in neural radiance fields (NeRF) in response to input of a five-dimensional parameter having the same elements as those of the parameter PRA. In other words, the transformation model 221 is trained to convert the five-dimensional parameter having the same elements as those of the parameter PRA into the four-dimensional parameter representing a color and volume density in the NeRF. The transformation model 221 can be built using, for instance, techniques disclosed in Non Patent Literature DH 1 and Ben Mildenhall, et. al, “NeRF: Representing Scenes as Neural Radiance Fields for View Synthesis” (hereinafter, referred to as Non Patent Literature DH 2). According to the present example embodiment, for instance, it is desirable to use, as learning data in a case where the transformation model 221 performs the learning, data including a plurality of lesion model images obtained by capturing, at a plurality of angles different from each other, a lesion model that imitates a shape or the like of an actual lesion. According to the present example embodiment, for instance, it is desirable to use, as the learning data in a case where the transformation model 221 performs the learning, data including a plurality of simulated images obtained by changing, at a plurality of angles different from each other, an angle of an actual lesion contained in an image. According to the present example embodiment, data including at least one lesion model image and at least one simulated image may be used as the learning data in a case where the transformation model 221 performs the learning.

The parameter conversion unit 22 acquires a four-dimensional parameter (R, G, B, σ) representing a color and volume density in the NeRF as a parameter PRB by inputting the parameter PRA(x, y, z, α, β) obtained by the parameter acquisition unit 21 to the transformation model 221, and outputs the acquired parameter PRB to the image generation unit 23.

The parameter conversion unit 22 can acquire a value of R of the parameter PRB as a pixel value representing a magnitude of a red component in the viewpoint position and the line-of-sight direction that correspond to the parameter PRA. The parameter conversion unit 22 can acquire a value of G of the parameter PRB as a pixel value representing a magnitude of a green component in the viewpoint position and the line-of-sight direction that correspond to the parameter PRA. The parameter conversion unit 22 can acquire a value of B of the parameter PRB as a pixel value representing a magnitude of a blue component in the viewpoint position and the line-of-sight direction that correspond to the parameter PRA. The parameter conversion unit 22 can acquire a value of σ of the parameter PRB as a density value representing volume density in the viewpoint position and the line-of-sight direction that correspond to the parameter PRA. The volume density described above can be rephrased as opacity.

As described above, the parameter conversion unit 22 can convert the parameter PRA into the parameter PRB by using the transformation model 221 trained to output the parameter representing the color and the volume density in response to the input of the parameter having the same elements as those of the parameter PRA.

The image generation unit 23 has a function of image generation means. The image generation unit 23 generates five arbitrary-viewpoint endoscopic images corresponding to five respective viewpoint positions different from each other by performing volume rendering processing conforming to a preset combination of an azimuth angle αp and an angle of elevation/depression βp, by using the parameter PRB obtained by the parameter conversion unit 22. The image generation unit 23 distributes, one by one, the five arbitrary-viewpoint endoscopic images generated as described above to the five inference processing units 24A to 24E. In other words, the image generation unit 23 outputs, one by one, the five arbitrary-viewpoint endoscopic images generated as described above to the five inference processing units 24A to 24E in an exclusive manner. The image generation unit 23 may use the technique disclosed in Non Patent literature DH 2 or may use another technique in a case where the above-described volume rendering processing is performed.

A specific example of the arbitrary-viewpoint endoscopic images generated by the processing of the image generation unit 23 will now be described.

The image generation unit 23 performs, for instance, volume rendering processing conforming to a combination of (αp, βp)=(0°, 0°) by using the parameter PRB obtained by the parameter conversion unit 22. According to such processing, the image generation unit 23 can generate an endoscopic image EGN in which the subject contained in the endoscopic image EG is viewed from the same position as the viewpoint position of the endoscopic image EG.

The image generation unit 23 performs, for instance, volume rendering processing conforming to a combination of (αp, βp)=(+10°, 0°) by using the parameter PRB obtained by the parameter conversion unit 22. According to such processing, the image generation unit 23 can generate an endoscopic image EGL in which the subject contained in the endoscopic image EG is viewed from a position shifted leftward with respect to the viewpoint position of the endoscopic image EG.

The image generation unit 23 performs, for instance, volume rendering processing conforming to a combination of (αp, βp)=(−10°, 0°) by using the parameter PRB obtained by the parameter conversion unit 22. According to such processing, the image generation unit 23 can generate an endoscopic image EGR in which the subject contained in the endoscopic image EG is viewed from a position shifted rightward with respect to the viewpoint position of the endoscopic image EG.

The image generation unit 23 performs, for instance, volume rendering processing conforming to a combination of (αp, βp)=(0°, +10°) by using the parameter PRB obtained by the parameter conversion unit 22. According to such processing, the image generation unit 23 can generate an endoscopic image EGD in which the subject contained in the endoscopic image EG is viewed from a position shifted downward with respect to the viewpoint position of the endoscopic image EG.

The image generation unit 23 performs, for instance, volume rendering processing conforming to a combination of (αp, βp)=(0°, −10°) by using the parameter PRB obtained by the parameter conversion unit 22. According to such processing, the image generation unit 23 can generate an endoscopic image EGU in which the subject contained in the endoscopic image EG is viewed from a position shifted upward with respect to the viewpoint position of the endoscopic image EG.

As described above, the image generation unit 23 can generate an image group containing the plurality of arbitrary-viewpoint endoscopic images corresponding to the respective viewpoint positions different from each other by using the parameter PRB that is obtained based on the endoscopic image EG containing the subject in the body cavity.

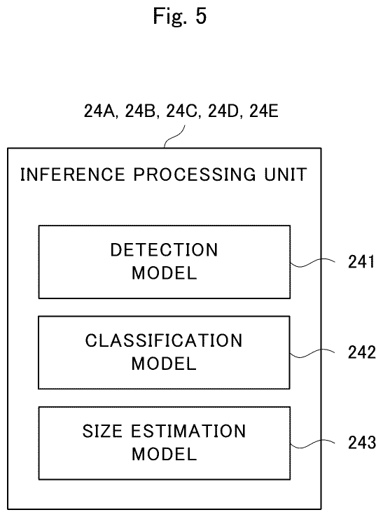

In the present example embodiment, description will be given taking as an example a case where the endoscopic image EGN is output to the inference processing unit 24A, the endoscopic image EGL is output to the inference processing unit 24B, the endoscopic image EGR is output to the inference processing unit 24C, the endoscopic image EGD is output to the inference processing unit 24D, and the endoscopic image EGU is output to the inference processing unit 24E. In the present example embodiment, as illustrated in FIG. 5, the five inference processing units 24A to 24E have the same function. Specifically, the five inference processing units 24A to 24E each have a function of inference means that performs inference processing based on the endoscopic image EGN, EGL, EGR, EGD, or EGU. Hence, the function of the inference processing unit 24A will be mainly described hereinafter, and description of the functions of the inference processing units 24B to 24E will be omitted as necessary. FIG. 5 is a diagram for describing a functional configuration example of the inference processing units.

The inference processing unit 24A is configured in such a way as to perform inference processing conforming to an input signal NS that is generated in the input unit 14, as processing for the endoscopic image EGN obtained from the image generation unit 23, and output an inference result ERN obtained by the inference processing to the inference result integration unit 25. Specifically, in a case where an input signal NS related to detection of the lesion is generated in the input unit 14, for instance, the inference processing unit 24A performs inference processing for detecting presence or absence of the lesion in the endoscopic image EGN. In a case where an input signal NS related to diagnosis of the lesion is generated in the input unit 14, for instance, the inference processing unit 24A performs inference processing for evaluating tumorigenicity and non-tumorigenicity of the lesion contained in the endoscopic image EGN. In a case where an input signal NS related to measurement of a size of the lesion is generated in the input unit 14, for instance, the inference processing unit 24A performs inference processing for estimating the size of the lesion contained in the endoscopic image EGN. As illustrated in FIG. 5, the inference processing unit 24A includes a detection model 241, a classification model 242, and a size estimation model 243.

The detection model 241 is configured as a machine learning model trained to perform the inference processing for detecting the presence or absence of the lesion in the endoscopic image input from the outside, and output an inference result obtained by the inference processing. According to the configuration of this detection model 241, for instance, it is possible to obtain an inference result capable of identifying the presence or absence of the lesion in the endoscopic image and a position of the lesion in the endoscopic image. According to the configuration of the detection model 241 as described above, for instance, it is possible to obtain an inference result in which a higher score is given in a case where the lesion is contained in the endoscopic image, and a lower score is given in a case where the lesion is not contained in the endoscopic image.

The classification model 242 is configured as a machine learning model trained to perform the inference processing for evaluating the tumorigenicity and the non-tumorigenicity of the lesion contained in the endoscopic image input from the outside, and output an inference result obtained by the inference processing. According to the configuration of this classification model 242, for instance, it is possible to obtain an inference result capable of identifying whether the lesion contained in the endoscopic image is tumorigenic or non-tumorigenic. According to the above-described configuration of the classification model 242, for instance, it is possible to obtain an inference result in which a sum of a score indicating the tumorigenicity of the lesion contained in the endoscopic image and a score indicating the non-tumorigenicity of the lesion is 1.

The size estimation model 243 is configured as a machine learning model trained to perform the inference processing for estimating the size of the lesion contained in the endoscopic image input from the outside, and output an inference result obtained by the inference processing. According to the configuration of the size estimation model 243, for instance, it is possible to obtain an inference result capable of identifying whether the lesion contained in the endoscopic image has a size larger than a predetermined size. According to the above-described configuration of the size estimation model 243, for instance, it is possible to obtain an inference result in which a higher score is given in a case where the size of the lesion contained in the endoscopic image is larger than the predetermined size, and a lower score is given in a case where the size of the lesion is equal to or smaller than the predetermined size. The above-described predetermined size is desirably set to 5 mm, for instance.

The inference processing unit 24B is configured in such a way as to perform inference processing conforming to an input signal NS that is generated in the input unit 14, as processing for the endoscopic image EGL obtained from the image generation unit 23, and output an inference result ERL obtained by the inference processing to the inference result integration unit 25. The inference processing unit 24B includes a detection model 241, a classification model 242, and a size estimation model 243 that are similar to those of the inference processing unit 24A. Hence, the inference processing unit 24B can perform the inference processing similar to that of the inference processing unit 24A in conformity with the input signal NS generated in the input unit 14.

The inference processing unit 24C is configured in such a way as to perform inference processing conforming to an input signal NS that is generated in the input unit 14, as processing for the endoscopic image EGR obtained from the image generation unit 23, and output an inference result ERR obtained by the inference processing to the inference result integration unit 25. The inference processing unit 24C includes a detection model 241, a classification model 242, and a size estimation model 243 that are similar to those of the inference processing unit 24A. Hence, the inference processing unit 24C can perform the inference processing similar to that of the inference processing unit 24A in conformity with the input signal NS generated in the input unit 14.

The inference processing unit 24D is configured in such a way as to perform inference processing conforming to an input signal NS that is generated in the input unit 14, as processing for the endoscopic image EGD obtained from the image generation unit 23, and output an inference result ERD obtained by the inference processing to the inference result integration unit 25. The inference processing unit 24D includes a detection model 241, a classification model 242, and a size estimation model 243 that are similar to those of the inference processing unit 24A. Hence, the inference processing unit 24D can perform the inference processing similar to that of the inference processing unit 24A in conformity with the input signal NS generated in the input unit 14.

The inference processing unit 24E is configured in such a way as to perform inference processing conforming to an input signal NS that is generated in the input unit 14, as processing for the endoscopic image EGU obtained from the image generation unit 23, and output an inference result ERU obtained by the inference processing to the inference result integration unit 25. The inference processing unit 24E includes a detection model 241, a classification model 242, and a size estimation model 243 that are similar to those of the inference processing unit 24A. Hence, the inference processing unit 24E can perform the inference processing similar to that of the inference processing unit 24A in conformity with the input signal NS generated in the input unit 14.

As described above, the inference processing units 24A to 24E can obtain five inference results by performing the inference processing individually on the five arbitrary-viewpoint endoscopic images.

The inference result integration unit 25 performs processing for integrating the inference result ERN output from the inference processing unit 24A, the inference result ERL output from the inference processing unit 24B, the inference result ERR output from the inference processing unit 24C, the inference result ERD output from the inference processing unit 24D, and the inference result ERU output from the inference processing unit 24E. The inference result integration unit 25 acquires an integrated inference result obtained by the above-described processing, as a final inference result ERF relevant to the endoscopic image EG, and outputs the acquired final estimation result ERF to an outside.

A specific example of the processing performed in the inference result integration unit 25 will now be described. Hereinafter, for convenience of description, the inference results ERN, ERL, ERR, ERD, and ERU are also referred to as five inference results.

The inference result integration unit 25 acquires, as the integrated inference result, an inference result that occupies a majority of the five inference results. Such processing of the inference result integration unit 25 can be dealt with as processing corresponding to hard voting.

Alternatively, the inference result integration unit 25 acquires an integrated estimation result based on a total value of scores included in the five inference results. Specifically, for instance, in a case where the five inference results are obtained by the inference processing using the detection models 241, the inference result integration unit 25 acquires an integrated estimation result according to whether the total value of the scores included in the five inference results is higher than a predetermined value. In a case where the five inference results are obtained by the inference processing using the classification models 242, for instance, the inference result integration unit 25 acquires an integrated estimation result based on a result obtained by comparing a total value of scores that are included in the five inference results and indicates the tumorigenicity of the lesion with a total value of scores that are included in the five inference results and indicates the non-tumorigenicity of the lesion. In a case where the five inference results are obtained by the inference processing using the size estimation models 243, for instance, the inference result integration unit 25 acquires an integrated estimation result according to whether a total value of scores included in the five inference results is higher than a predetermined value. These pieces of processing of the inference result integration unit 25 can be dealt with as processing corresponding to soft voting.

According to the processing described in the above specific example, in a case where the five inference results are obtained by the inference processing using the detection models 241, for instance, the inference result integration unit 25 can obtain, as the final inference result ERF relevant to the endoscopic image EG, one of an inference result that the lesion is present in the endoscopic image EG and an inference result that the lesion is not present in the endoscopic image EG. According to the processing described in the above specific example, in a case where the five inference results are obtained by the inference processing using the classification models 242, for instance, the inference result integration unit 25 can obtain, as the final inference result ERF relevant to the endoscopic image EG, one of an inference result that the lesion contained in the endoscopic image EG is tumorigenic and an inference result that the lesion contained in the endoscopic image EG is non-tumorigenic. According to the processing described in the above specific example, in a case where the five inference results are obtained by the inference processing using the size estimation models 243, for instance, the inference result integration unit 25 can obtain, as the final inference result ERF relevant to the endoscopic image EG, one of an inference result that the lesion contained in the endoscopic image EG has the size larger than the predetermined size and an inference result that the lesion contained in the endoscopic image EG has the size equal to or smaller than the predetermined size.

As described above, the inference result integration unit 25 can acquire the final inference result relevant to the endoscopic image EG by integrating a plurality of the inference results obtained by performing the inference processing individually on the plurality of arbitrary-viewpoint endoscopic images. As described above, the inference result integration unit 25 can integrate the plurality of inference results by performing the processing corresponding to any one of the hard voting and the soft voting.

According to the present example embodiment, processing for presenting the final estimation result ERF to the operator may be performed in the endoscopic examination support device 1. Specifically, for instance, in a case where the final inference result ERF is obtained through the inference processing using the detection models 241, a frame indicating the position of the lesion in the endoscopic image EG may be displayed. In a case where the final inference result ERF is obtained through the inference processing using the classification models 242, for instance, character information indicating a state of the lesion contained in the endoscopic image EG may be displayed. In a case where the final inference result ERF is obtained through the inference processing using the size estimation models 243, for instance, character information indicating the size of the lesion contained in the endoscopic image EG may be displayed.

According to the present example embodiment, the image generation unit 23 may be configured in such a way as to generate p (p≥2) arbitrary-viewpoint endoscopic images corresponding to p viewpoint positions different from each other. In such a case, in order to perform the inference processing individually on the p arbitrary-viewpoint endoscopic images, p inference processing units having the same function as that of the inference processing unit 24A may be provided in the endoscopic examination support device 1.

According to the present example embodiment, the endoscopic image EG, instead of the endoscopic image EGN, may be input to the inference processing unit 24A. In such a case, the image generation unit 23 may generate, by using the parameter PRB, an image group containing (p−1) arbitrary-viewpoint endoscopic image(s) corresponding to respective (each) (p−1) viewpoint position(s) different from the viewpoint position of the endoscopic image EG. In the above case, in order to perform the inference processing individually on the endoscopic image EG and the (p−1) arbitrary-viewpoint endoscopic image(s), p inference processing units having the same function as that of the inference processing unit 24A may be provided in the endoscopic examination support device 1. In the above case, the p inference processing units can perform the inference processing based on the endoscopic image EG and the (p−1) arbitrary-viewpoint endoscopic image(s). In the above case, the inference result integration unit 25 can acquire a final inference result relevant to the endoscopic image EG by integrating a plurality of inference results obtained by performing the inference processing individually on the endoscopic image EG and the (p−1) arbitrary-viewpoint endoscopic image(s).

According to the present example embodiment, in a case where the processing corresponding to the hard voting is performed in the inference result integration unit 25, it is desirable to input an odd number of inference results to the inference result integration unit 25.

### Processing Flow

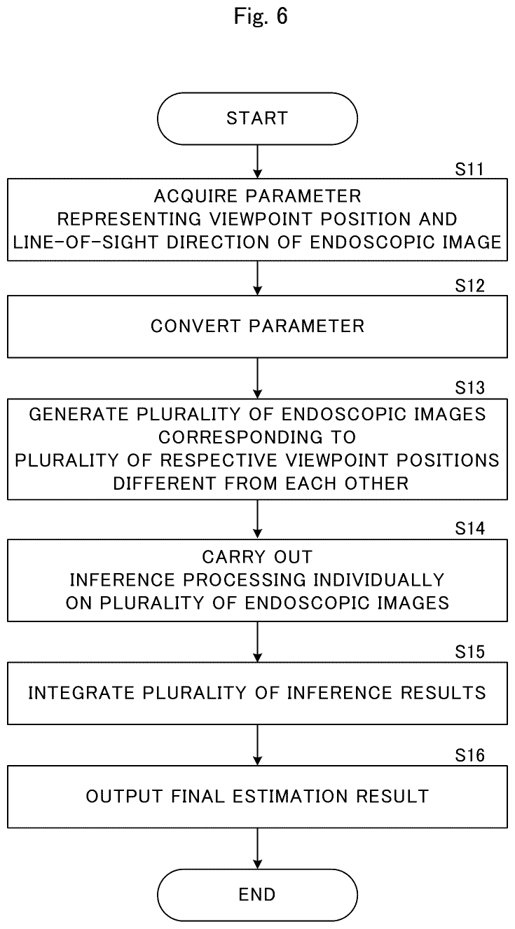

Next, a flow of the processing performed in the endoscopic examination support device 1 will be described. FIG. 6 is a flowchart illustrating an example of the processing performed in the endoscopic examination support device according to the present disclosure.

First, the endoscopic examination support device 1 acquires the five-dimensional parameter representing the viewpoint position and the line-of-sight direction that correspond to each pixel of the endoscopic image (step S11).

Next, the endoscopic examination support device 1 converts the five-dimensional parameter obtained in step S11 into the four-dimensional parameter representing the color and the volume density in the NeRF by performing the processing using the transformation model 221 (step S12).

Subsequently, the endoscopic examination support device 1 generates the plurality of endoscopic images corresponding to the plurality of viewpoint positions different from each other by performing the volume rendering processing using the four-dimensional parameter obtained in step S12 (step S13). In step S13, the endoscopic examination support device 1 may generate an endoscopic image at a viewpoint position same as the viewpoint position of the endoscopic image being the processing target in step S11.

Subsequently, the endoscopic examination support device 1 acquires the plurality of inference results relevant to the plurality of respective endoscopic images by performing the inference processing individually on the plurality of endoscopic images generated in step S13 (step S14). According to the present example embodiment, the endoscopic examination support device 1 can perform the processing using any one of the detection model 241, the classification model 242, and the size estimation model 243, as the inference processing in step S14.

Subsequently, the endoscopic examination support device 1 performs the processing for integrating the plurality of inference results obtained by the inference processing in step S14 (step S15). According to the present example embodiment, the endoscopic examination support device 1 can selectively perform the processing corresponding to any one of the hard voting and the soft voting as the processing of step S15.

Finally, the endoscopic examination support device 1 acquires the integrated estimation result obtained in step S15 as the final inference result relevant to the endoscopic image being the processing target in step S11, and outputs the acquired final estimation result (step S16).

As described above, according to the present example embodiment, it is possible to acquire the plurality of inference results by performing the inference processing individually on the plurality of endoscopic images corresponding to the plurality of respective viewpoint positions different from each other, and acquire the final inference result by integrating the plurality of inference results. Hence, according to the present example embodiment, for instance, it is possible to improve accuracy of the inference results obtained by performing the inference processing related to the detection of the presence or absence of the lesion in the endoscopic image. According to the present example embodiment, for instance, it is possible to improve accuracy of the inference results obtained by performing the inference processing related to the diagnosis (classification) of the lesion contained in the endoscopic image. Further, for instance, it is possible to improve accuracy of the inference results obtained by performing the inference processing related to the size estimation of the lesion contained in the endoscopic image. Thus, according to the present example embodiment, it is possible to improve accuracy of information related to the lesion obtained from the endoscopic image.

### Second Example Embodiment

Next, a second example embodiment of the present disclosure will be described. Hereinafter, for the sake of simplicity, a specific description regarding part to which the processing described above and the like is applicable will be omitted as necessary.

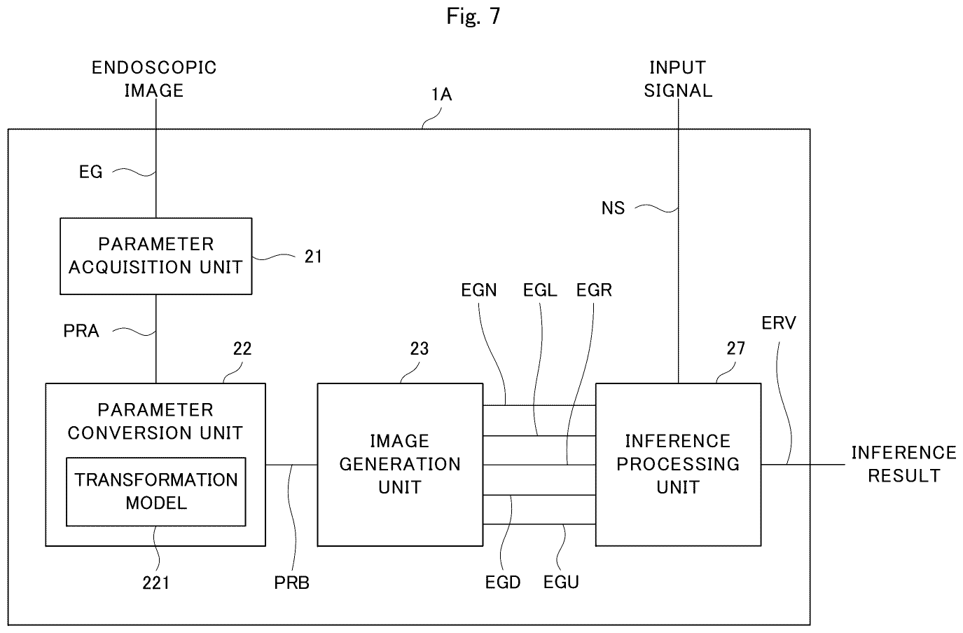

FIG. 7 is a block diagram illustrating another functional configuration example of the endoscopic examination support device according to the present disclosure. An endoscopic examination support device 1A has a hardware configuration similar to that of the endoscopic examination support device 1. As illustrated in FIG. 7, the endoscopic examination support device 1A includes a parameter acquisition unit 21, a parameter conversion unit 22, an image generation unit 23, and an inference processing unit 27. Hereinafter, for convenience of description, endoscopic images EGN, EGL, EGR, EGD, and EGU are also referred to as five endoscopic images.

The image generation unit 23 generates the five endoscopic images using a method similar to the method described in the first example embodiment, and outputs the generated five endoscopic images to the inference processing unit 27.

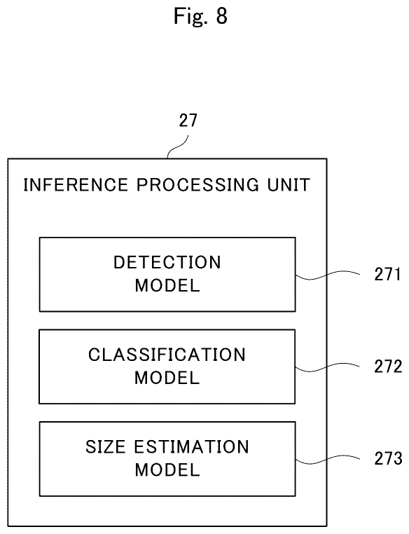

The inference processing unit 27 has a function of inference means. The inference processing unit 27 is configured in such a way as to perform inference processing conforming to an input signal NS that is generated in the input unit 14, as processing for the five endoscopic images obtained from the image generation unit 23, and output a comprehensive inference result ERV obtained by the inference processing to the outside. Specifically, in a case where an input signal NS related to detection of a lesion is generated in the input unit 14, for instance, the inference processing unit 27 performs inference processing for detecting presence or absence of the lesion in each of the five endoscopic images. In a case where an input signal NS related to diagnosis of the lesion is generated in the input unit 14, for instance, the inference processing unit 27 performs inference processing for evaluating tumorigenicity and non-tumorigenicity of the lesion contained in each of the five endoscopic images. In a case where an input signal NS related to measurement of a size of the lesion is generated in the input unit 14, for instance, the inference processing unit 27 performs inference processing for estimating the size of the lesion contained in each of the five endoscopic images. As illustrated in FIG. 8, the inference processing unit 27 includes a detection model 271, a classification model 272, and a size estimation model 273. FIG. 8 is a diagram for describing another functional configuration example of the inference processing unit.

The detection model 271 is configured as a machine learning model trained to perform the inference processing for detecting the presence or absence of the lesion in each of a plurality of the endoscopic images input simultaneously from the outside, and output a comprehensive inference result obtained by the inference processing. According to the configuration of this detection model 271, it is possible to obtain, as the comprehensive inference result, one of an inference result that the lesion is present in each of the endoscopic images and an inference result that the lesion is not present in each of the endoscopic images.

The classification model 272 is configured as a machine learning model trained to perform the inference processing for evaluating the tumorigenicity and the non-tumorigenicity of the lesion contained in each of the plurality of endoscopic images input simultaneously from the outside, and output a comprehensive inference result obtained by the inference processing. According to the configuration of this classification model 272, it is possible to obtain, as the comprehensive inference result, one of an inference result that the lesion contained in each of the endoscopic images is tumorigenic and an inference result that the lesion contained in each of the endoscopic images is non-tumorigenic.

The size estimation model 273 is configured as a machine learning model trained to perform the inference processing for estimating the size of the lesion contained in each of the plurality of endoscopic images input simultaneously from the outside, and output a comprehensive inference result obtained by the inference processing. According to the configuration of this size estimation model 273, it is possible to obtain, as the comprehensive inference result, one of an inference result that the lesion contained in each of the endoscopic images has a size larger than a predetermined size and an inference result that the lesion contained in each of the endoscopic images has a size equal to or smaller than the predetermined size. The above-described predetermined size is desirably set to 5 mm, for instance.

According to the processing as described above, in a case where the inference processing using the detection model 271 is performed, for instance, the inference processing unit 27 can obtain, as a comprehensive inference result ERV relevant to an endoscopic image EG, one of an inference result that the lesion is present in the endoscopic image EG and an inference result that the lesion is not present in the endoscopic image EG. According to the processing described above, in a case where the inference processing using the classification model 272 is performed, for instance, the inference processing unit 27 can obtain, as a comprehensive inference result ERV relevant to the endoscopic image EG, one of an inference result that the lesion contained in the endoscopic image EG is tumorigenic and an inference result that the lesion contained in the endoscopic image EG is non-tumorigenic. According to the processing described above, in a case where the inference processing using the size estimation model 273 is performed, for instance, the inference processing unit 27 can obtain, as a comprehensive inference result ERV relevant to the endoscopic image EG, one of an inference result that the lesion contained in the endoscopic image EG has the size larger than the predetermined size and an inference result that the lesion contained in the endoscopic image EG has the size equal to or smaller than the predetermined size.

According to the present example embodiment, processing for presenting the comprehensive inference result ERV to the operator may be performed in the endoscopic examination support device 1. Specifically, for instance, in a case where the comprehensive inference result ERV is obtained through the inference processing using the detection model 271, a frame indicating a position of the lesion in the endoscopic image EG may be displayed. In a case where the comprehensive inference result ERV is obtained through the inference processing using the classification model 272, for instance, character information indicating a state of the lesion contained in the endoscopic image EG may be displayed. In a case where the comprehensive inference result ERV is obtained through the inference processing using the size estimation model 273, for instance, character information indicating the size of the lesion contained in the endoscopic image EG may be displayed.

According to the present example embodiment, the endoscopic image EG, instead of the endoscopic image EGN, may be input to the inference processing unit 27. In such a case, the image generation unit 23 may generate, by using the parameter PRB, q (q≥1) arbitrary-viewpoint endoscopic image(s) corresponding to q viewpoint position(s) different from a viewpoint position of the endoscopic image EG. In the above case, the inference processing unit 27 can obtain a comprehensive inference result by performing the inference processing based on the endoscopic image EG and the q arbitrary-viewpoint endoscopic image(s).

### Processing Flow

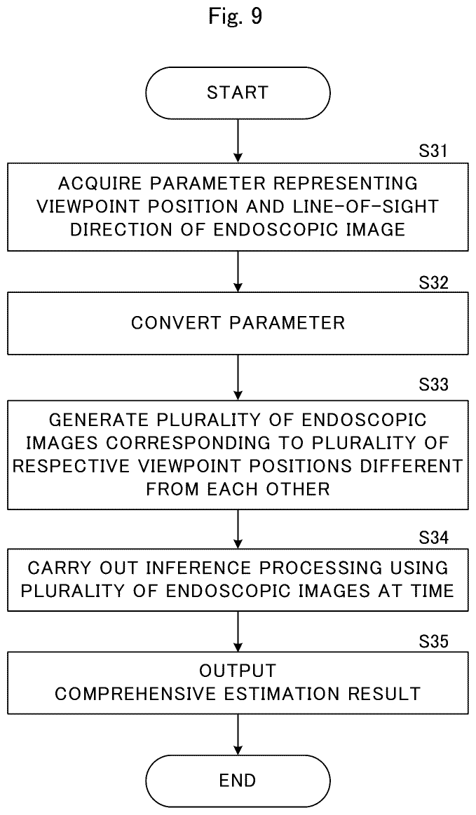

Next, a flow of the processing performed in the endoscopic examination support device 1A will be described. FIG. 9 is a flowchart illustrating another example of the processing performed in the endoscopic examination support device according to the present disclosure.

The endoscopic examination support device 1A performs, as processing in steps S31 to S33, processing similar to steps S11 to S13 of the first example embodiment.

Subsequently, the endoscopic examination support device 1A acquires the comprehensive inference result relevant to the endoscopic image being the processing target in step S31 by performing the inference processing using a plurality of the endoscopic images generated in step S33 at a time (step S34). According to the present example embodiment, the endoscopic examination support device 1A can perform the processing using any one of the detection model 271, the classification model 272, and the size estimation model 273, as the inference processing in step S34.

Finally, the endoscopic examination support device 1A outputs the comprehensive estimation result obtained in step S34 (step S35).

As described above, according to the present example embodiment, it is possible to acquire the comprehensive inference result by performing the inference processing using, at a time, the plurality of endoscopic images corresponding to a plurality of respective viewpoint positions different from each other. Hence, according to the present example embodiment, for instance, it is possible to improve accuracy of the inference results obtained by performing the inference processing related to the detection of the presence or absence of the lesion in the endoscopic image. According to the present example embodiment, for instance, it is possible to improve accuracy of the inference results obtained by performing the inference processing related to the diagnosis (classification) of the lesion contained in the endoscopic image. Further, for instance, it is possible to improve accuracy of the inference results obtained by performing the inference processing related to the size estimation of the lesion contained in the endoscopic image. Thus, according to the present example embodiment, it is possible to improve accuracy of information related to the lesion obtained from the endoscopic image.

### Third Example Embodiment

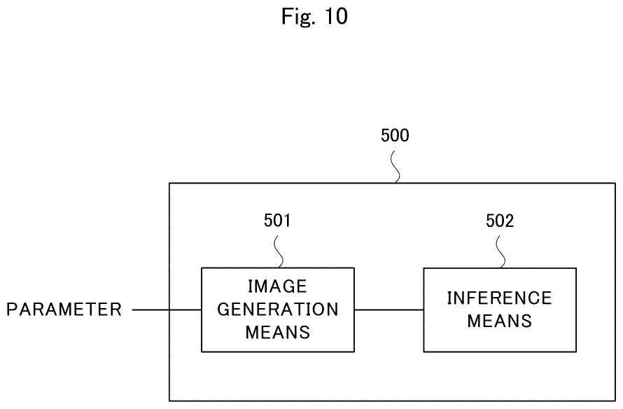

FIG. 10 is a block diagram illustrating still another functional configuration example of the endoscopic examination support device according to the present disclosure.

An endoscopic examination support device 500 according to the present example embodiment has a hardware configuration similar to that of the endoscopic examination support device 1. The endoscopic examination support device 500 includes image generation means 501 and inference means 502.

The image generation means 501 can be achieved by using, for instance, the function included in the image generation unit 23 of the first example embodiment. The inference means 502 can be achieved by using, for instance, the functions included in the inference processing units 24A to 24E of the first example embodiment or the inference processing unit 27 of the second example embodiment.

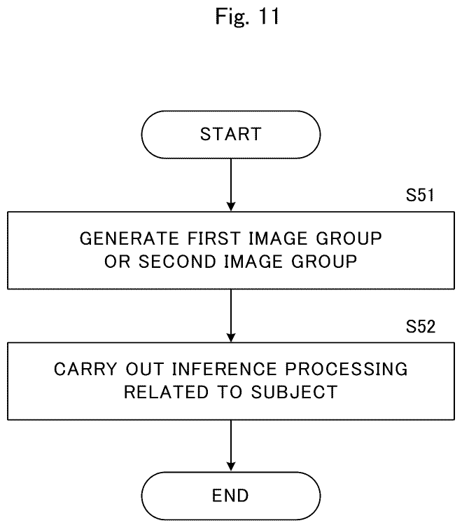

FIG. 11 is a flowchart illustrating still another example of the processing performed in the endoscopic examination support device according to the present disclosure.

The image generation means 501 generates a first image group containing a plurality of arbitrary-viewpoint endoscopic images corresponding to respective viewpoint positions different from each other by using a parameter that is obtained based on an endoscopic image containing a subject in a body cavity (step S51). Alternatively, the image generation means 501 generates, by using the parameter that is obtained based on the endoscopic image containing the subject in the body cavity, a second image group containing at least one arbitrary-viewpoint endoscopic image corresponding to a viewpoint position different from a viewpoint position of the endoscopic image (step S51).

The inference means 502 performs inference processing related to the subject based on the first image group (step S52). Alternatively, the inference means 502 performs the inference processing related to the subject based on the endoscopic image and the second image group (step S52).

According to the present example embodiment, it is possible to improve accuracy of information related to a lesion obtained from the endoscopic image.

Some or all of the above example embodiments may be described as the following Supplementary Notes, but are not limited to the following.

### Supplementary Note 1

An endoscopic examination support device comprising:
image generation means configured to, by using a parameter obtained based on an endoscopic image containing a subject in a body cavity, generate a first image group containing a plurality of arbitrary-viewpoint endoscopic images corresponding to respective viewpoint positions different from each other, or generate a second image group containing at least one arbitrary-viewpoint endoscopic image corresponding to a viewpoint position different from a viewpoint position of the endoscopic image; and
inference means configured to perform inference processing related to the subject based on the first image group or based on the endoscopic image and the second image group.

### Supplementary Note 2

The endoscopic examination support device according to claim 1, further comprising integration means configured to acquire a final inference result relevant to the endoscopic image by integrating a plurality of inference results obtained by performing the inference processing individually on the plurality of arbitrary-viewpoint endoscopic images contained in the first image group.

### Supplementary Note 3

The endoscopic examination support device according to claim 2, wherein the integration means integrates the plurality of inference results by performing processing corresponding to any one of hard voting and soft voting.

### Supplementary Note 4

The endoscopic examination support device according to claim 1, further comprising integration means configured to acquire a final inference result relevant to the endoscopic image by integrating a plurality of inference results obtained by performing the inference processing individually on the at least one arbitrary-viewpoint endoscopic image contained in the second image group.

### Supplementary Note 5

The endoscopic examination support device according to claim 4, wherein the integration means integrates the plurality of inference results by performing processing corresponding to any one of hard voting and soft voting.

### Supplementary Note 6

The endoscopic examination support device according to claim 1, further comprising:
parameter acquisition means configured to acquire a first parameter representing a viewpoint position and a line-of-sight direction corresponding to each pixel of the endoscopic image; and
parameter conversion means configured to convert the first parameter into a second parameter,
wherein the image generation means generates the first image group or the second image group by using the second parameter.

### Supplementary Note 7

The endoscopic examination support device according to claim 6, wherein the parameter conversion means converts the first parameter into the second parameter by using a machine learning model trained to output a parameter representing a color and volume density in response to input of a parameter having an element same as that of the first parameter.

### Supplementary Note 8

An endoscopic examination support method comprising:
by using a parameter obtained based on an endoscopic image containing a subject in a body cavity, generating a first image group containing a plurality of arbitrary-viewpoint endoscopic images corresponding to respective viewpoint positions different from each other, or generating a second image group containing at least one arbitrary-viewpoint endoscopic image corresponding to a viewpoint position different from a viewpoint position of the endoscopic image; and
performing inference related to the subject based on the first image group or based on the endoscopic image and the second image group.

### Supplementary Note 9

A recording medium storing a program, the program causing a computer to perform a process comprising:
processing of, by using a parameter obtained based on an endoscopic image containing a subject in a body cavity, generating a first image group containing a plurality of arbitrary-viewpoint endoscopic images corresponding to respective viewpoint positions different from each other, or generating a second image group containing at least one arbitrary-viewpoint endoscopic image corresponding to a viewpoint position different from a viewpoint position of the endoscopic image; and
processing of performing inference related to the subject based on the first image group or based on the endoscopic image and the second image group.

While the present disclosure has been particularly shown and described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims. And each embodiment can be appropriately combined with other embodiments.

### DESCRIPTION OF SYMBOLS

1, 1A Endoscopic examination support device
2 Display device
3 Endoscopic scope
11 Processor
12 Memory
13 Interface
21 Parameter acquisition unit
22 Parameter conversion unit
23 Image generation unit
24A, 24B, 24C, 24D, 24E, 27 Inference processing unit
25 Inference result integration unit
100 Endoscopic examination system

What is claimed is:

1. An endoscopic examination support device comprising:
at least one memory configured to store instructions; and
at least one processor configured to execute the instructions to:
by using a parameter obtained based on an endoscopic image containing a subject in a body cavity, generate a first image group containing a plurality of arbitrary-viewpoint endoscopic images corresponding to respective viewpoint positions different from each other, or generate a second image group containing at least one arbitrary-viewpoint endoscopic image corresponding to a viewpoint position different from a viewpoint position of the endoscopic image; and
inference processing related to the subject based on the first image group or based on the endoscopic image and the second image group.

2. The endoscopic examination support device according to claim 1, wherein the processor is further configured to acquire a final inference result relevant to the endoscopic image by integrating a plurality of inference results obtained by performing the inference processing individually on the plurality of arbitrary-viewpoint endoscopic images contained in the first image group.

3. The endoscopic examination support device according to claim 2, wherein the processor integrates the plurality of inference results by performing processing corresponding to any one of hard voting and soft voting.

4. The endoscopic examination support device according to claim 1, wherein the processor is further configured to acquire a final inference result relevant to the endoscopic image by integrating a plurality of inference results obtained by performing the inference processing individually on the at least one arbitrary-viewpoint endoscopic image contained in the second image group.

5. The endoscopic examination support device according to claim 4, wherein the processor integrates the plurality of inference results by performing processing corresponding to any one of hard voting and soft voting.

6. The endoscopic examination support device according to claim 1, wherein the processor is further configured to
acquire a first parameter representing a viewpoint position and a line-of-sight direction corresponding to each pixel of the endoscopic image; and
convert the first parameter into a second parameter,
wherein the processor generates the first image group or the second image group by using the second parameter.

7. The endoscopic examination support device according to claim 6, wherein the processor converts the first parameter into the second parameter by using a machine learning model trained to output a parameter representing a color and volume density in response to input of a parameter having an element same as that of the first parameter.

8. An endoscopic examination support method performed by a computer, the method comprising:
by using a parameter obtained based on an endoscopic image containing a subject in a body cavity, generating a first image group containing a plurality of arbitrary-viewpoint endoscopic images corresponding to respective viewpoint positions different from each other, or generating a second image group containing at least one arbitrary-viewpoint endoscopic image corresponding to a viewpoint position different from a viewpoint position of the endoscopic image; and
performing inference related to the subject based on the first image group or based on the endoscopic image and the second image group.

9. A non-transitory computer readable recording medium storing a program, the program causing a computer to perform a process comprising:
processing of, by using a parameter obtained based on an endoscopic image containing a subject in a body cavity, generating a first image group containing a plurality of arbitrary-viewpoint endoscopic images corresponding to respective viewpoint positions different from each other, or generating a second image group containing at least one arbitrary-viewpoint endoscopic image corresponding to a viewpoint position different from a viewpoint position of the endoscopic image; and
processing of performing inference related to the subject based on the first image group or based on the endoscopic image and the second image group.
